(19) United States
(12) Patent Application Publication
(10) Pub. No.: US 2024/0324919 A1
(43) Pub. Date: Oct. 3, 2024

(54) Capillary Blood Collection Device

(71) Applicant: Becton, Dickinson and Company, Franklin Lakes, NJ (US)

(72) Inventors: Anthony V. Torris, Mercer Island, WA (US); Vlad Yakhnich, Park Ridge, NJ (US); Scott Wentzell, Suffern, NY (US); Kishore K. Bokka Srinivasa Rao, Ridgewood, NJ (US); Daniel J. Marchiarullo, Morris Plains, NJ (US)

(21) Appl. No.: 18/573,686

(22) PCT Filed: Jun. 23, 2022
(86) PCT No.: PCT/US22/34626

§ 371 Date: Dec. 22, 2023

Related U.S. Application Data
(60) Provisional application No. 63/216,239, filed on Jun. 29, 2021.

(51) Int. Cl. A61B 5/15; A61B 5/151

(52) U.S. Cl. CPC A61B 5/150022; A61B 5/150343; A61B 5/151

(57) ABSTRACT

A device for obtaining a blood sample may include a holder for receiving a sample source, the holder having an actuation portion and a port; a blood collector attachment removably connected to the holder; and a collection container removably connectable to the blood collector attachment, the container defining a collection cavity, wherein the blood collector attachment comprises a post member configured to direct the blood sample from a patient's finger to the collection container.

### CROSS-REFERENCE TO RELATED APPLICATION

The present application claims priority to U.S. Provisional Application Ser. No. 63/216,239, filed Jun. 29, 2021, entitled “Capillary Blood Collection Device”, the entire disclosure of which is hereby incorporated by reference in its' entirety.

### BACKGROUND OF THE INVENTION

### Field of the Invention

The present disclosure relates generally to a device for obtaining a biological sample. More particularly, the present disclosure relates to an integrated finger-based capillary blood collection device with the ability to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medial field is capillary blood collection which is often done to collect blood samples for testing. Certain diseases, such as diabetes, require that the patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member such as a needle, or a sharp-edged member such as a blade, that is used to make a quick puncture wound or incision in the patient's skin in order to provide a small outflow of blood. It is often physiologically and psychologically difficult for many people to prick their own finger with a hand-held needle or blade. As a result, lancet devices have evolved into automatic devices that puncture or cut the skin of the patient upon the actuation of a triggering mechanism. In some devices, the needle or blade is kept in a standby position until it is triggered by the user, who may be a medical professional in charge of drawing blood from the patient, or the patient himself or herself. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releases the lever from interference engagement with the lancet structure. The blood sample that is received is then collected and/or tested. This testing can be done by a Point-of-Care (POC) testing device or it can be collected and sent to a testing facility.

Currently, capillary blood collection workflow is a complex multi-step process requiring high skill level. The multi-step nature of this process introduces several variables that could cause sample quality issues such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

Capillary collection blood draws are typically performed by health care workers either using their fingers to manually squeeze the tissue around the puncture site or by a device using vacuum pressure to pull blood from the site.

Manually squeezing the collection site is a highly technique dependent process that leads to very large variation in success rate and sample quality (as measured by hemolysis−blood cell rupture). Health care workers typically adjust the pressure and rate at which they squeeze to compensate for patient-dependent differences in blood flow. Squeezing harder helps blood flow more quickly but also increases hemolysis. The location of squeezing also varies between health care workers depending on personal preference, experience, and hand fatigue. Some workers may even perform a process called “milking” of fingers, where they apply pressure starting at the base of the finger and slide towards the tip of finger. This process is discouraged as leading to poor sample quality by domestic and international health organizations.

Vacuum-powered devices standardize the pressure and technique of blood flow, but are typically plagued by poor overall blood flow. The maximum pressure than can be applied is limited by the difference between atmospheric pressure and absolute vacuum (˜14 psi), and devices only operate at a fraction of absolute vacuum. For reference, grip strength of men and women range from 50-100 lbs. on average, illustrating why manual methods are instead affected by hemolysis rather than flow. Vacuum methods also apply consistent pressure, limiting the ability of the tissue to replenish with blood.

Thus, there is a need in the art for a device that has the ability to lance and squeeze the finger, collect the sample, stabilize the sample, and subsequently dispense the sample in a controlled manner. There is also a need in the art for a device that simplifies and streamlines the capillary blood collection by eliminating workflow variabilities which are typically associated with low sample quality including hemolysis and micro-clots. There is still a further need in the art for a closed system collection and transfer that eliminate blood exposure and device reuse. There is still a further need in the art for a device that: (1) introduces flexibility in the accommodation of different capillary blood collection and transfer container; (2) has the capability to generate high quality uniformly mixed/stabilized capillary blood samples; (3) has the capability to generate on-board plasma from capillary plasma samples; (4) has the capability to collect large capillary blood samples (>50-500 μL) at reduced pain; (5) contains a unique sample identifier that is paired with patient information at the time of collection; (6) has the capability to collect capillary blood and perform on-board diagnostics; and (7) has multiple collection ports to collect a blood sample into different containers having the same or different anticoagulants. There is a further need in the art for a capillary blood collection device that includes a standardized and controlled location of applied pressure, an applied pressure that is high enough for adequate blood flow but below hemolysis thresholds, a defined rhythmic application of pressure rather than consistent pressure to allow blood to replenish in the finger, increasing average blood flow rate, and a reduced user fatigue by lowering maximum applied force by the operator.

### SUMMARY OF THE INVENTION

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

In one embodiment of the present disclosure, a device for obtaining a blood sample may include a holder for receiving a sample source, the holder having an actuation portion and a port; a blood collector attachment removably connected to the holder; and a collection container removably connectable to the blood collector attachment, the container defining a collection cavity, wherein the blood collector attachment comprises a post member configured to direct the blood sample from a patient's finger to the collection container.

In one embodiment of the present disclosure, the post member may be positioned within the blood collector attachment so as to be directly beneath the patient's finger to make contact with the blood sample drawn from the patient's finger. A top edge of the post member may be shorter than a top edge of the blood collector attachment. The post member may be positioned within the blood collector attachment so as not to contact the patient's finger when held in the holder. The blood collector attachment may include a guided flow path connected to the post member for directing the blood sample from the post member to the collection container. The blood collector attachment may include a transition path provided between the post member and the guided flow path. The transition path may have a curvature. A coating may be applied to the post member to prevent adhesion of the blood sample to the post member. A bottom edge of the blood collector attachment may have a curvature to direct the blood sample from the blood collector attachment to the collection container. At least one vent may be defined in an upper surface of the blood collector attachment. The blood collector attachment may define an open channel flow path between the patient's finger and the collection container. The open channel flow path may include at least one rib.

In one embodiment of the present disclosure, a device for obtaining a blood sample may include a holder for receiving a sample source, the holder having an actuation portion and a port; and a blood collector attachment removably connected to the holder; and wherein the blood collector attachment comprises a post member configured to direct the blood sample from a patient's finger to a collection container.

In one embodiment of the present disclosure, the post member may be positioned within the blood collector attachment so as to be directly beneath the patient's finger to make contact with the blood sample drawn from the patient's finger. A top edge of the post member may be shorter than a top edge of the blood collector attachment. The post member may be positioned within the blood collector attachment so as not to contact the patient's finger when held in the holder. The blood collector attachment may include a guided flow path connected to the post member for directing the blood sample from the post member to the collection container. The blood collector attachment may include a transition path provided between the post member and the guided flow path. The transition path may have a curvature. A coating may be applied to the post member to prevent adhesion of the blood sample to the post member. A bottom edge of the blood collector attachment may have a curvature to direct the blood sample from the blood collector attachment to the collection container. At least one vent may be defined in an upper surface of the blood collector attachment. The blood collector attachment may define an open channel flow path between the patient's finger and the collection container. The open channel flow path may include at least one rib.

In one embodiment of the present disclosure, a device for obtaining a blood sample may include a blood collector attachment configured to be removably connected to a holder and a collection container; and wherein the blood collector attachment comprises a post member configured to direct the blood sample from a patient's finger to the collection container.

The present invention is also disclosed in the following clauses:

Clause 1: A device for obtaining a blood sample, the device comprising: a holder for receiving a sample source, the holder having an actuation portion and a port; a blood collector attachment removably connected to the holder; and a collection container removably connectable to the blood collector attachment, the container defining a collection cavity, wherein the blood collector attachment comprises a post member configured to direct the blood sample from a patient's finger to the collection container.

Clause 2: The device of Clause 1, wherein the post member is positioned within the blood collector attachment so as to be directly beneath the patient's finger to make contact with the blood sample drawn from the patient's finger.

Clause 3: The device of Clause 1 or 2, wherein a top edge of the post member is shorter than a top edge of the blood collector attachment.

Clause 4: The device of any of Clauses 1-3, wherein the post member is positioned within the blood collector attachment so as not to contact the patient's finger when held in the holder.

Clause 5: The device of any of Clauses 1-4, wherein the blood collector attachment further comprises a guided flow path connected to the post member for directing the blood sample from the post member to the collection container.

Clause 6: The device of Clause 5, wherein the blood collector attachment further comprises a transition path provided between the post member and the guided flow path.

Clause 7: The device of Clause 6, wherein the transition path has a curvature.

Clause 8: The device of any of Clauses 1-7, wherein a coating is applied to at least one of the blood collector attachment and the collection container to reduce adhesion of at least part of the blood sample thereto.

Clause 9: The device of any of Clauses 1-8, wherein the coating is applied to the post member.

Clause 10: The device of any of Clauses 1-9, wherein the coating is a protein coating.

Clause 11: The device of any of Clauses 1-10, wherein the protein coating comprises Bovine Serum albumin.

Clause 12: The device of any of Clauses 1-11, wherein the coating comprises EDTA.

Clause 13: The device of any of Clauses 1-12, wherein a bottom edge of the blood collector attachment has a curvature to direct the blood sample from the blood collector attachment to the collection container.

Clause 14: The device of any of Clauses 1-13, wherein at least one vent is defined in an upper surface of the blood collector attachment.

Clause 15: The device of Clauses 1-14, wherein the blood collector attachment defines an open channel flow path between the patient's finger and the collection container.

Clause 16: The device of Clause 15, wherein the open channel flow path comprises at least one rib.

Clause 17: A device for obtaining a blood sample, the device comprising: a holder for receiving a sample source, the holder having an actuation portion and a port; and a blood collector attachment removably connected to the holder; and wherein the blood collector attachment comprises a post member configured to direct the blood sample from a patient's finger to a collection container.

Clause 18: The device of Clause 17, wherein the post member is positioned within the blood collector attachment so as to be directly beneath the patient's finger to make contact with the blood sample drawn from the patient's finger.

Clause 19: The device of Clause 17 or 18, wherein a top edge of the post member is shorter than a top edge of the blood collector attachment.

Clause 20: The device of any of Clauses 17-19, wherein the post member is positioned within the blood collector attachment so as not to contact the patient's finger when held in the holder.

Clause 21: The device of any of Clauses 17-20, wherein the blood collector attachment further comprises a guided flow path connected to the post member for directing the blood sample from the post member to the collection container.

Clause 22: The device of Clause 21, wherein the blood collector attachment further comprises a transition path provided between the post member and the guided flow path.

Clause 23: The device of Clause 22, wherein the transition path has a curvature.

Clause 24: The device of any of Clauses 13-19, wherein a coating is applied to the post member to prevent adhesion of the blood sample to the post member.

Clause 25: The device of Clause 24, wherein a coating is applied to at least one of the blood collector attachment and the collection container to reduce adhesion of at least part of the blood sample thereto.

Clause 26: The device of any of Clauses 24-25, wherein the coating is applied to the post member.

Clause 27: The device of any of Clauses 24-26, wherein the coating is a protein coating.

Clause 28: The device of any of Clauses 24-27, wherein the protein coating comprises Bovine Serum albumin.

Clause 29: The device of any of Clauses 24-28, wherein the coating comprises EDTA.

Clause 30: The device of any of Clauses 17-29, wherein a bottom edge of the blood collector attachment has a curvature to direct the blood sample from the blood collector attachment to the collection container.

Clause 31: The device of any of Clauses 17-30, wherein at least one vent is defined in an upper surface of the blood collector attachment.

Clause 32: The device of any of Clauses 13-31, wherein the blood collector attachment defines an open channel flow path between the patient's finger and the collection container.

Clause 33: The device of Clause 32, wherein the open channel flow path comprises at least one rib.

Clause 34: A device for obtaining a blood sample, the device comprising: a blood collector attachment configured to be removably connected to a holder and a collection container; and wherein the blood collector attachment comprises a post member configured to direct the blood sample from a patient's finger to the collection container.

### BRIEF DESCRIPTION OF THE DRAWINGS

FIG. 1 is a perspective view of a holder in accordance with an embodiment of the present invention.

FIG. 2A is a cross-sectional view of a device for obtaining a blood sample from a patient's finger and a lancet in accordance with another embodiment of the present disclosure.

FIG. 2B is a perspective view of a device for obtaining a blood sample from a patient's finger and a sample collection container in accordance with another embodiment of the present disclosure.

FIG. 3 is a perspective cross sectional view of a device for obtaining a blood sample from a patient's finger and a collection container in accordance with another embodiment of the present disclosure.

FIG. 4 is a schematic illustration of a holder according to an embodiment of the present disclosure in an active state.

FIG. 5 is a schematic illustration of the holder of FIG. 4 in which a blood sample is being drawn from a patient's finger.

FIG. 6 is a bottom perspective view of a blood collector attachment having a capillary channel according to one embodiment of the present disclosure.

FIG. 7 is a schematic illustration of a blood sample flow path of the blood collector attachment of FIG. 6.

FIG. 8 is a bottom perspective view of a blood collector attachment having an open channel according to one embodiment of the present disclosure.

FIG. 9 is a schematic illustration of a blood sample flow path of the blood collector attachment of FIG. 8.

FIG. 10 is a schematic illustration of the blood collector attachment of FIG. 8 including a rib in the open channel.

FIG. 11 is a perspective view of a blood collector attachment removably attached to a collection container according to one embodiment of the present disclosure.

FIG. 12 is a perspective view of a “mini” blood collector attachment according to one embodiment of the present disclosure.

FIG. 13 is a side view of the “mini” blood collector attachment of FIG. 12.

FIG. 14 is a bottom perspective view of the “mini” blood collector attachment of FIG. 12.

FIG. 15 is a side cross sectional view of the “mini” blood collector attachment of FIG. 12.

FIG. 16 is a perspective cross sectional view of a blood collector attachment removably attached to a collection container according to one embodiment of the present disclosure.

FIG. 17 is a perspective cross sectional view of a “mini” blood collector attachment removably attached to a collection container according to one embodiment of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

Blood collection is fundamentally driven by pressure-driven flow. Devices or techniques either reduce the pressure outside the blood vessel (vacuum-powered flow) or increase the pressure inside the vessels. Both approaches increase the difference between the blood vessel pressure and external pressure, and increase the flow rate from inside the vessel to outside where the collection container is present. The location of squeezing can also be critical, as soft tissues (e.g. fat, skin, and musculature) are perfused with blood while hard tissues and joints are poorly perfused or are too mechanically stable to compress without patient pain.

Red blood cells (RBCs) are subject to hemolysis during collection. Hemolysis (RBC destruction) contaminates samples for diagnostic analysis, both by spilling cell contents into the liquid serum of the sample and by coloring the serum red via hemoglobin and interfering with colorimetric reactions. The amount of hemolysis during collection is driven by shear-mediated destruction of the cells due to flow rate and flow path as well as pressure-driven hemolysis where physical compression of tissues and vessels can damage cells. Hemolysis can therefore be controlled by ensuring that applied pressures and flows are not too high in any of the locations of the finger being squeezed.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

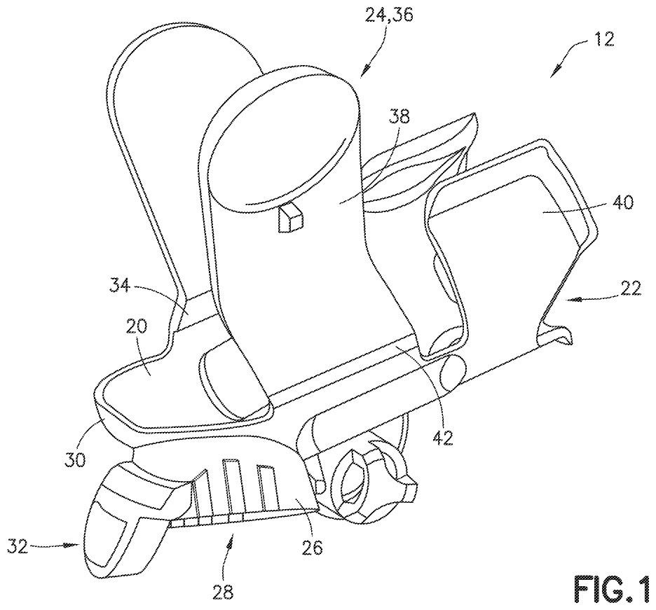

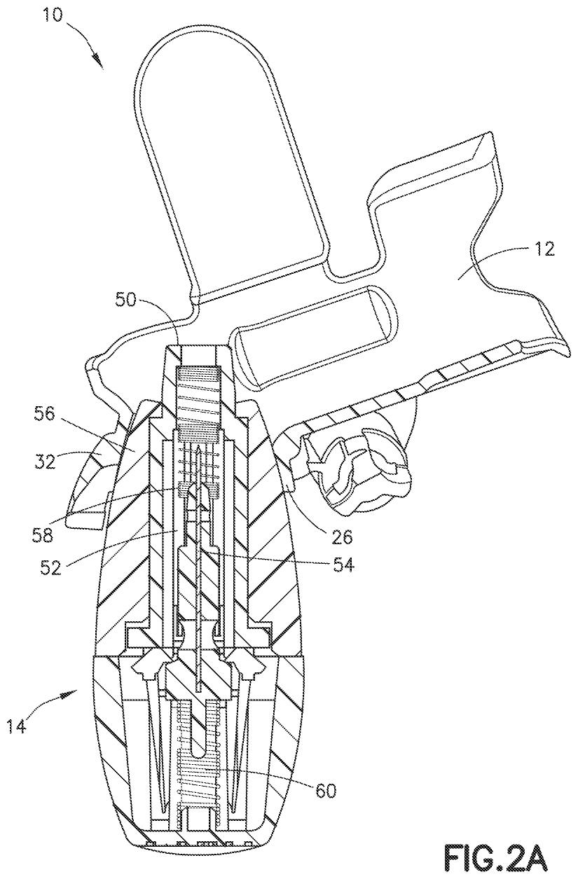

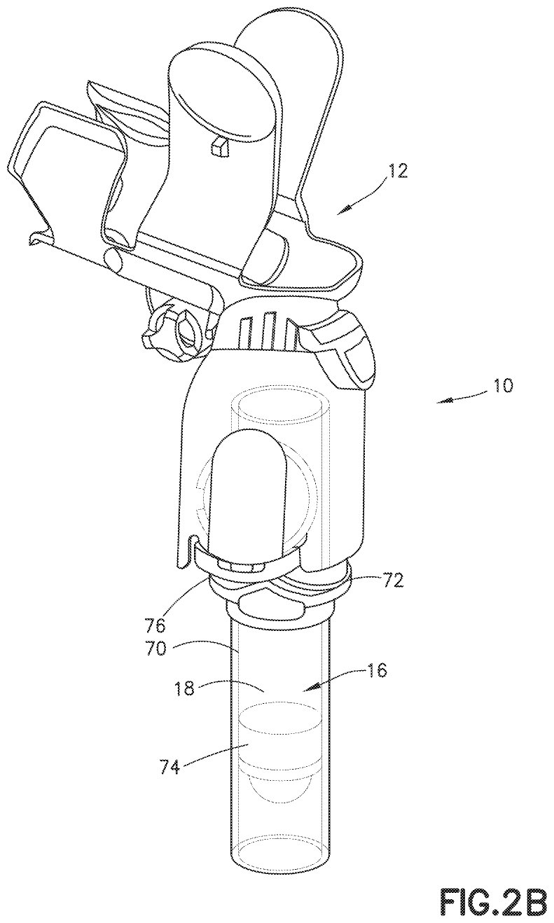

Referring to FIGS. 1 and 2A, in an exemplary embodiment, a device 10 of the present disclosure includes discrete components, e.g., a holder 12 (as shown in FIG. 1), a lancet housing or lancet 14 (as shown in FIG. 2A), and a collection container 16 (as shown in FIG. 2B). In another exemplary embodiment, a semi-integrated device of the present disclosure may include an at-angle flow and include an integrated lancet housing and collection container which can be connected with a separate holder. In another exemplary embodiment, a semi-integrated device of the present disclosure may have an in-line flow and include an integrated lancet housing and collection container which can be connected with a separate holder. In another exemplary embodiment, an integrated device of the present disclosure may have an at-angle flow and include an integrated holder, lancet housing, and collection container. In another exemplary embodiment, an integrated device of the present disclosure may have an in-line flow and include an integrated holder, lancet housing, and collection container.

Referring to FIG. 1, an exemplary embodiment of a holder 12 of the present disclosure that is able to receive a sample source, e.g., a finger 19, for supplying a biological sample, such as a blood sample 18, is shown and described. A holder 12 of the present disclosure generally includes a finger receiving portion 20 having a first opening 22 (FIG. 1), an actuation portion 24, a port 26 having a second opening 28, and a finger end guard 30. In one embodiment, the finger end guard 30 provides a stop portion for properly aligning and securing a finger 19 within the holder 12. The finger end guard 30 further assists in ensuring the patient's finger 19 is placed at a proper position within the finger receiving portion 20 so that applied pressure to the patient's finger 19 will result in adequate blood flow.

The first opening 22 of the finger receiving portion 20 is configured for receiving a sample source, e.g., a finger 19, for supplying a biological sample, such as a blood sample 18 (shown in FIG. 2B). It can be appreciated that the sample source could include other parts of the body capable of fitting within the first opening 22. The port 26 is in communication with the finger receiving portion 20. For example, with a finger 19 received within the holder 12, the port 26 is in communication with a portion of the finger 19. A holder 12 of the present disclosure can be sized to accommodate all finger sizes.

The second opening 28 of the port 26 is configured for receiving a lancet housing 14 and a collection container 16 as described in more detail below. In one embodiment, the port 26 includes a locking portion 32 for securely receiving the lancet housing 14 and the collection container 16 within the port 26.

In one embodiment, the actuation portion 24 is transitionable between a first position in which the holder 12 defines a first diameter and a second position which the holder 12 defines a second diameter, wherein the second diameter is less than the first diameter. In one embodiment, the actuation portion 24 is transitionable between a first position in which the holder 12 defines a first elliptical shape, and a second position in which the holder 12 defines a second elliptical shape, wherein the first elliptical shape is different than the second elliptical shape. In this manner, with the holder 12 in the second position with a reduced diameter, a portion of the holder 12 contacts the sample source and the actuation portion 24 of the holder 12 is able to pump and/or extract blood 18 as described in more detail below.

Referring to FIG. 1, in one embodiment, the actuation portion 24 includes a contact member 34. With the actuation portion 24 in the first position, the contact member 34 is in a disengaged position, i.e., the contact member 34 is provided in a first position with respect to a sample source, e.g., the finger 19, such that the contact member 34 may be in slight contact therewith. With the actuation portion 24 in the second position, the contact member 34 is in an engaged position, i.e., the contact member 34 is provided in a second position with respect to the sample source, e.g., the finger 19, such that the contact member 34 is in an applied pressure contact with the finger 19, and the actuation portion 24 of the holder 12 is able to pump and/or extract blood 18. For example, with the contact member 34 in the engaged position, the contact member 34 exerts a pressure on the sample source.

Referring to FIG. 1, in one embodiment, the actuation portion 24 includes a pumping member 36 for applying pressure to the sample source, e.g., the finger 19. In one embodiment, the pumping member 36 comprises a pair of opposed tabs or wings 38. In such an embodiment, each tab 38 may include a contact member 34. In one embodiment, the holder 12 includes a living hinge portion 42. The living hinge portion 42 allows a user to squeeze the wings 38 between a first position (passive state) and a second position (active state). The use of the tabs or wings 38 to draw blood 18 out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12 while flexing to accommodate the largest patient finger within a holder 12 without blood occlusion.

Advantageously, the holder 12 of the present disclosure allows a user to repeatedly squeeze and release the wings 38 to pump and/or extract blood 18 from a finger 19 until a desired amount of blood 18 is filled in a collection container 16. The wings 38 are configured to flex to maintain gentle contact with a range of patient finger sizes that may be used with the holder 12 and to retain the holder 12 on the patient's finger 19.

Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Referring to FIG. 1, in one embodiment, the holder 12 includes a stability extension portion 40. This provides additional support for the holder 12 to be securely placed onto a finger 19. In one embodiment, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

In one embodiment, the finger receiving portion 20 is formed of a flexible material. In some embodiments, the finger receiving portion 20 and the port 26 are formed from a flexible material.

A device 10 for obtaining a blood sample 18 of the present disclosure includes a lancet housing or lancet 14 that is removably connectable to a port 26 of a holder 12. Referring to FIG. 2A, in one embodiment, the lancet housing 14 includes an inlet or opening 50, an interior 52, a puncturing element 54, an engagement portion 56, a retractable mechanism 58, and a drive spring 60. In one embodiment, the puncturing element 54 is moveable between a pre-actuated position wherein the puncturing element 54 is retained within the interior 52 of the lancet housing 14 and a puncturing position wherein at least a portion of the puncturing element 54 extends through the inlet 50 of the lancet housing 14 to lance a portion of a finger 19.

In one embodiment, the lancet 14 of the present disclosure is a contact activated lancet and may be constructed in accordance with the features disclosed in U.S. Patent Application Publication No. 2006/0052809 filed May 6, 2005, entitled “Contact Activated Lancet Device”, and commonly assigned with the present application, the entire disclosure of which is hereby expressly incorporated herein by reference thereto.

In one embodiment, the lancet housing 14 may be a separate component from the holder 12 and the collection container 16. In some embodiments, the collection container 16 and the lancet housing 14 form a single component that is removably connectable to the port 26 of the holder 12. In some embodiments, the collection container 16, the lancet housing 14, and the holder 12 form a single component.

Referring to FIG. 2A, in one embodiment, with the holder 12 and the lancet housing 14 being separate components, the lancet housing 14 is removably connectable to the port 26 of the holder 12. In such an embodiment, the lancet housing 14 includes an engagement portion 56. Referring to FIG. 2A, in one embodiment, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. In some embodiments, the port 26 of the holder 12 includes a plurality of ribs for securing and locking the lancet 14 or the collection container 16 in the port 26.

To activate the lancet 14, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

In one embodiment, the lancet 14 includes a drive spring 60 disposed within the interior 52 of the lancet housing 14 for biasing the puncturing element 54 toward the puncturing position. After puncturing, the puncturing element 54 is immediately retracted and safely secured within the interior 52 of the lancet housing 14.

In one embodiment, the lancet 14 of the present disclosure is used to lance the skin of a finger 19 and then a blood sample 18 is squeezed into a collection container 16 as described in more detail below.

In one embodiment, the lancet housing 14 of the present disclosure is used to lance the skin of a finger 19 along a lance path and then a blood sample 18 flows down a blood flow path at an angle to the lance path as described in more detail below.

In one embodiment, the lancet 14 includes a hollow needle. In such an embodiment, the lancet housing 14 of the present disclosure is used to lance the skin of a finger 19 along a lance path and then a blood sample 18 flows along a parallel blood flow path through the hollow needle.

As shown in FIG. 2B, a device 10 for obtaining a blood sample 18 of the present disclosure includes a collection container 16 that is removably connectable to the port 26 of the holder 12. The collection container 16 defines a collection cavity 70 for receiving a blood sample 18, a container engagement portion 72, a blood collector portion 74, and a cap or septum 76. Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

In one embodiment, the collection container 16 may be a separate component from the holder 12 and the lancet housing 14. In some embodiments, the collection container 16 and the lancet housing 14 form a single component that is removably connectable to the port 26 of the holder 12. In some embodiments, the collection container 16, the lancet housing 14, and the holder 12 form a single component.

In one embodiment, with the holder 12 and the collection container 16 being separate components, the container 16 is removably connectable to the port 26 of the holder 12. In such an embodiment, the container 16 includes a container engagement portion 72. In one embodiment, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

It can be appreciated that several types of collection containers 16 can be used with the device 10 of the present disclosure. It can also be appreciated that the collection container 16 can be associated with a separate dispensing unit or the collection container 16 can include an integral dispensing portion for dispensing the blood 18 to a testing device.

Referring to FIG. 1, use of a device 10 of the present disclosure having discrete components, e.g., a holder 12, a lancet housing or lancet 14, and a collection container 16, will now be described.

Referring to FIG. 1, first a desired finger 19 is cleaned and a holder 12 having an appropriate size for the desired finger 19 is selected and placed onto the finger 19 securely. Next, referring to FIG. 2A, a lancet housing 14 is connected to the port 26 of the holder 12. As discussed above, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 (FIG. 2A) of the lancet housing 14 can be activated to lance or puncture a sample source, e.g., a finger 19. With the lancet 14 connected to the port 26 of the holder 12, the lancet 14 is in communication with the finger 19.

When it is desired to activate the lancet 14 to lance the skin of a finger 19, the lancet 14 is pushed against a finger 19 to activate a retractable mechanism 58 (FIG. 2A) of the lancet 14 to lance a finger 19. The lancet 14 of the present disclosure consistently delivers correct lancing depth and a pre-defined lancing location, thus ensuring a sufficient sample volume.

After the finger 19 is lanced to create blood 18 flow from the finger 19, the lancet 14 is removed from the holder 12 and the collection container 16 is pushed into the port 26 of the holder 12. Referring to FIG. 2B, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

Referring to FIG. 1, with the container 16 properly secured to the holder 12 for collection of a blood sample 18, a user is able to repeatedly squeeze and release the wings 38 of the holder 12 to pump and/or extract blood 18 from a finger 19 until a desired amount of blood 18 is filled in a collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

For example, referring to FIG. 1, in one embodiment, the actuation portion 24 includes a contact member 34. With the actuation portion 24 in the first position, the contact member 34 is in a disengaged position, i.e., the contact member 34 is in the first position with respect to the sample source, e.g., the finger 19. With the actuation portion 24 in the second position, the contact member 34 is in an engaged position, i.e., the contact member 34 is in the second position and in applied pressure contact with a sample source, e.g., the finger 19, and the actuation portion 24 of the holder 12 is able to pump and/or extract blood 18. For example, with the contact member 34 in the engaged position, the contact member 34 exerts a pressure on the sample source.

Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

The devices of the present disclosure are compatible with any known testing device, whether the testing device is off-site or a point-of-care testing device. Various point-of-care testing devices are known in the art. Such point-of-care testing devices include test strips, glass slides, diagnostic cartridges, or other testing devices for testing and analysis. Test strips, glass slides, and diagnostic cartridges are point-of-care testing devices that receive a blood sample and test that blood for one or more physiological and biochemical states. There are many point-of-care devices that use cartridge based architecture to analyze very small amounts of blood bedside without the need to send the sample to a lab for analysis. This saves time in getting results over the long run, but creates a different set of challenges versus the highly routine lab environment. Examples of such testing cartridges include the i-STAT® testing cartridge from the Abbot group of companies. Testing cartridges such as the i-STAT® cartridges may be used to test for a variety of conditions including the presence of chemicals and electrolytes, hematology, blood gas concentrations, coagulation, or cardiac markers. The results of tests using such cartridges are quickly provided to the clinician.

The collection container 16 may also contain a sample stabilizer, e.g., an anticoagulant, to stabilize a blood sample 18 and/or a component of a blood sample 18 disposed therein. The collection container 16 may also include at least one fill line(s) corresponding to a predetermined volume of sample. The collection container may also indicate/meter a collected volume of blood.

Any of the devices for obtaining a blood sample of the present disclosure can be used as a self-standing disposable device and/or in association with an external power source for pain reduction control. For example, a portion of holder 12 may include embedded electrodes which receive a signal from an external pain control module to deliver at least one of heat, vibration, or transcutaneous electrical nerve stimulation (TENS) for pain reduction control. The devices for obtaining a blood sample of the present disclosure may also include various options for on-board plasma separation. The devices for obtaining a blood sample of the present disclosure may also include a unique sample identifier that can be paired with patient information at the time of collection. The devices for obtaining a blood sample of the present disclosure may also include on-board diagnostic feedback at the time of collection. A device for obtaining a blood sample of the present disclosure may also allow for dual collection, e.g., the collection of two samples into two separate containers, using multiple collection ports which enable the collection of multiple samples from the same source and treating the samples with different sample stabilizers, such as anticoagulants.

A device for obtaining a blood sample of the present disclosure significantly simplifies and de-skills large volume capillary collection from a finger relative to the conventional capillary collection using lancet and capillary tube. The devices of the present disclosure eliminate blood exposure and prevents device reuse.

The devices for obtaining a blood sample of the present disclosure simplify, deskill, and streamline the collection process. This is all achieved by a self-contained closed system device which after it is placed onto a finger will provide lancing, blood extraction, stabilization, and containment functions, all in one unit.

The devices for obtaining a blood sample of the present disclosure may be associated with a self-standing unit that provides automated pumping, controlled finger squeezing, and automated sample labeling and processing.

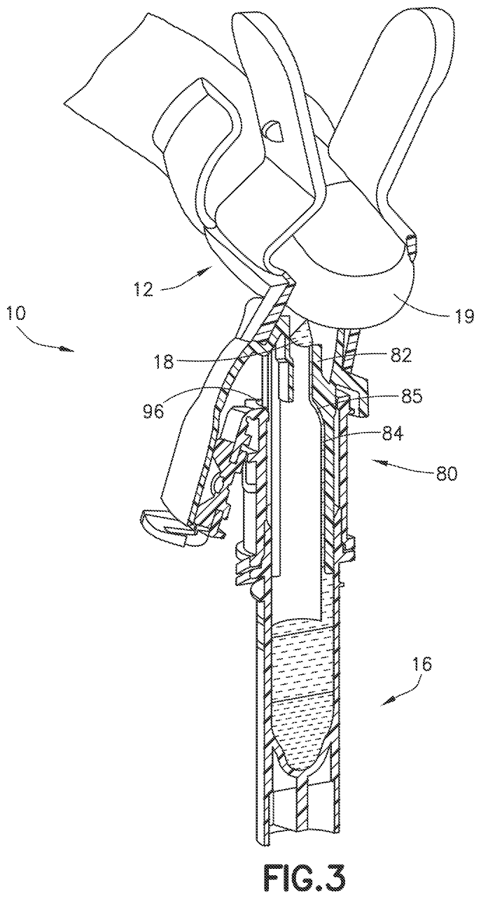

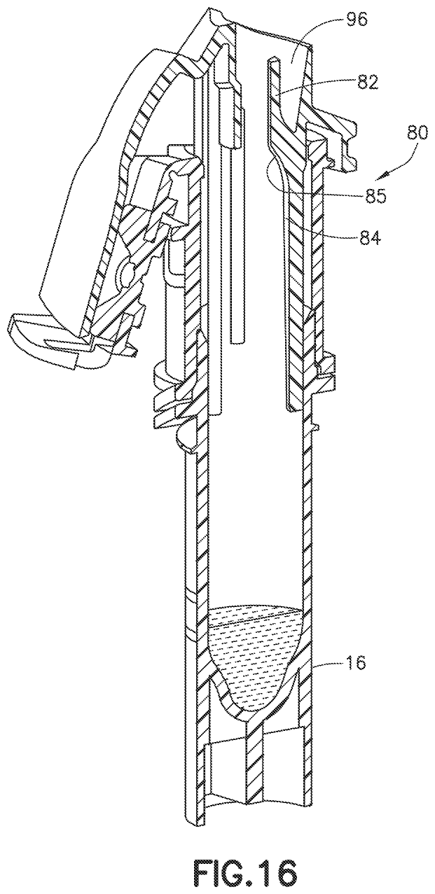

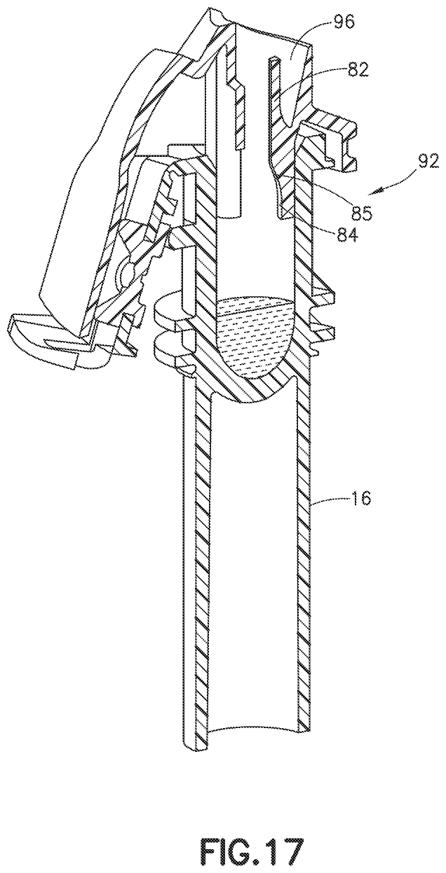

With reference to FIGS. 3 and 16, according to one embodiment of the present disclosure, a blood collector attachment 80 used in connection with the holder 12 and the collection container 16 is shown and described. The blood collector attachment 80 is configured to be removably connected to the port 26 of the holder 12 and the container engagement portion 72 of the collection container 16. The blood collector attachment 80 is configured to transfer the blood sample 18 from the patient's finger 19 to the collection container 16. The blood collector attachment 80 relies on gravity, geometric properties, and surface properties to transfer the blood sample 18 from the patient's finger 19 to the collection container 16. The flow path principles used in the design of the blood collector attachment 80 are not restricted to this collection device 10 and could be applicable to other capillary blood collection devices where a blood sample needs to be transferred from a punctured skin into a blood collection tube. As shown in FIG. 17, according to another embodiment of the present disclosure, a “mini” blood collector attachment 92 is shown. This “mini” blood collector attachment 92 may be substantially similar to the blood collector attachment 80, but has smaller dimensions and smaller surface areas.

The blood collector attachment 80 provides at least two primary functions. First, the blood collector attachment 80 provides a closed system for blood collection that minimizes the exposure of blood to the person or user (e.g healthcare professionals or patients themselves) collecting the blood sample 18 at the same time minimizing blood exposure to the patient. Second, the blood collector attachment 80 delivers the blood sample 18 from the patient's finger 19 to the collection container 16 with an improved sample quality.

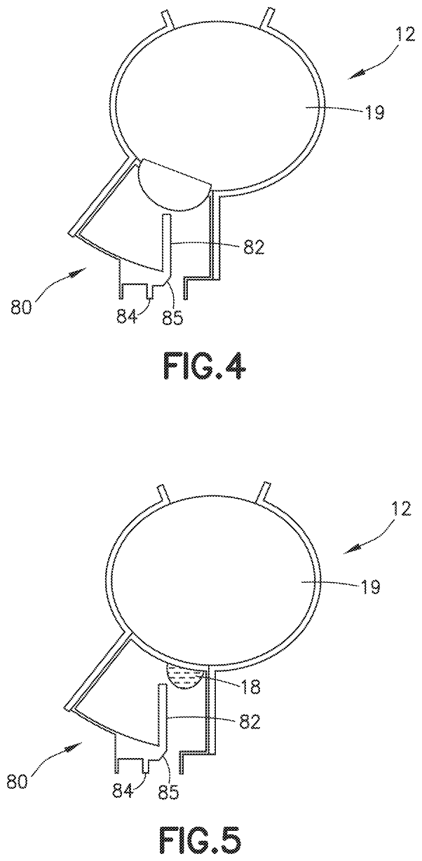

With continued reference to FIG. 3, in one embodiment of the present disclosure, the blood collector attachment 80 may include a post member 82 that extends from an inner surface of the blood collector attachment 80 towards the port 26 of the holder 12. In one embodiment, the post member 82 is positioned in a cavity defined by the blood collector attachment 80 and is positioned beneath the portion of the patient's finger 19 from which the blood sample 18 is withdrawn. As shown in FIGS. 4 and 5, the post member 82 provided in the blood collector attachment 80 provides a surface for a first drop of blood from the blood sample 18 coming out of the wound in the patient's finger 19 to establish a flow path with an inner surface of the blood collector attachment 80. As shown in FIG. 4, the height/location of the post member 82 may be optimized to ensure the post member 82 does not come in contact with the open wound in the patient's finger 19 during collection of the blood sample 18 to minimize the risk of infection. As shown in FIG. 5, at the same time, the position of the post member 82 is close enough to the wound in the patient's finger 19 to establish contact with the first drop of blood coming out of the wound. The outer wall of the blood collector attachment 80 should be higher than the top end of the post member 82 so the blood sample 18 is directed downwardly after making contact with the post member 82, instead of leaking out of the blood collector attachment 80.

As shown in FIG. 3, after the first drop of blood comes into contact with the post member 82, a guided flow path 84 provided on an inner surface of the blood collector attachment 80 directs the blood sample 18 to continue to flow down the post member 82 and into the collection container 16. The guided flow path 84 minimizes the smearing of the blood sample 18 on the collection container 16 and reduces blood hang up in the collection container 16, which also reduces the dead volume in the collection container 16. In one embodiment, the guided flow path 84 may extend substantially parallel with a longitudinal axis of the collection container 16. In one embodiment, a curved transition path 85 is provided between the post member 82 and the guided flow path 84. The curved transition path 85 may extend outwardly from the post member 82 towards the guided flow path 84.

In one embodiment of the present disclosure, several different factors assist in ensuring the blood sample 18 flows along the guided flow path 84 into the collection container 16. The material of the guided flow path 84 or a surfactant applied to the guided flow path 84 may assist in moving the blood sample 18 along the guided flow path 84. Hydrophilic surface properties for the guided flow path 84 may be desirable, which can be achieved through multiple ways such as a hydrophilic material for the guided flow path 84, a copolymer in the guided flow path 84 that provides hydrophilic surface properties, and a surface treatment with chemicals like surfactant that provide blood wicking properties to the surface of the guided flow path 84. Both hydrophobic and hydrophilic materials may help to different levels in minimizing the platelet adhesion to the blood collector attachment 80. Changing the surface properties of the blood collector attachment 80 to extreme hydrophobic and hydrophilic type surface properties may reduce the platelet adhesion to a great extent. A surfactant coating may be used on the blood collector attachment 80 to reduce cell hang up and adhesion of cellular particles to the blood collector attachment surfaces. A lubricant may be used on the surfaces of the blood collector attachment 80 to repel the blood sample 18 from the surface of the blood collector attachment 80, thereby reducing cell adhesion and hang up on the surfaces of the blood collector attachment 80. A protein coating, such as BSA (Bovine Serum albumin), may also act as a barrier to cell adhesion on the surfaces of the blood collector attachment 80, thereby reducing platelet adhesion to the surface of blood collector attachment 80. Coating the surfaces of the blood collector attachment 80 with an anticoagulant (EDTA), may introduce the additive sooner to the blood sample 18 and may help stabilize the blood sample 18 faster. This approach may prevent platelet aggregation, clumping and formation of micro clots and/or clots, thereby reducing platelet adhesion to the surface of the blood collector attachment 80.

In one example, a gravitational force ensures the blood sample 18 continues to run along the guided flow path 84 into the collection container 16. In addition to the gravitational force on the blood sample 18, a surface tension established between the blood sample 18 and the guided flow path 84 assists in routing the blood sample 18 into the collection container 16. The flow phenomenon may be further enhanced by surface modifications. Hydrophilic surface characteristics help improve flow, which may be achieved by using hydrophilic materials or plasma treating the material or coating with additives like surfactants. Geometric features in the blood collector attachment 90 may act as obstacles to guide the flow and restrict the smearing of blood.

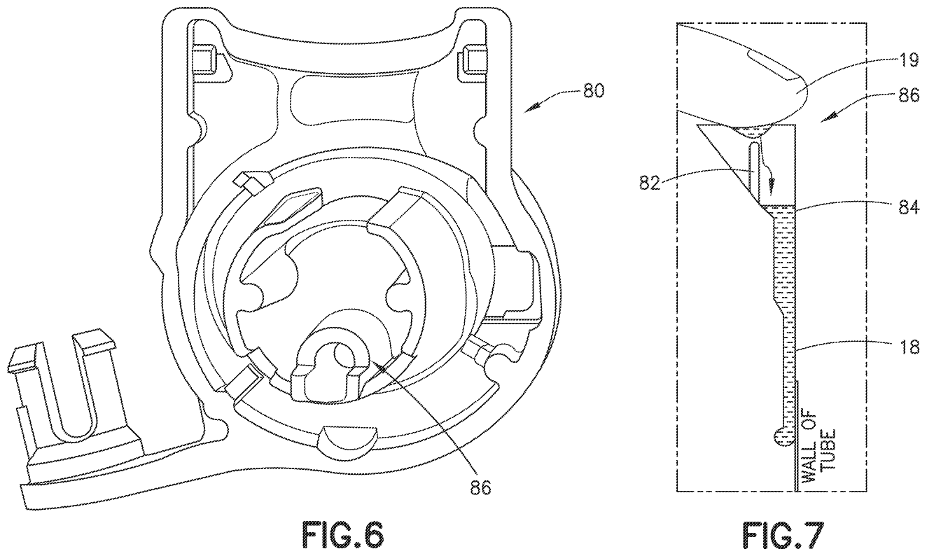

Several features are provided in the blood collector attachment 80 to improve the blood sample 18 quality that is delivered to the collection container 16. The main blood sample quality attribute that is affected by the blood collector attachment 80 is the platelet count in the blood sample 18. The platelets tend to stick to the surface of the blood collector attachment 80, and activated platelets coming out of an open wound have a higher tendency to stick to surfaces. Therefore, the blood collector attachment 80 of the present disclosure includes several features that help reduce the platelet adhesion on the blood collector attachment 80 surfaces. As shown in FIGS. 6-10, in several embodiments of the present disclosure, a flow geometry of the blood collector attachment 80 may be adjusted to reduce platelet adhesion. In FIGS. 6 and 7, a capillary channel configuration may be provided on the blood collector attachment 80 to utilize a closed channel flow path 86 for the blood sample 18 through the blood collector attachment 80. In this closed channel configuration, the flow path for the blood sample 18 is reduced to ensure uniform flow of the blood sample 18 without platelet adhesion on the surfaces of the blood collector attachment 80. The capillary channel provides a faster flow through higher surface tension forces but with risk of blood pooling and increased dead volume.

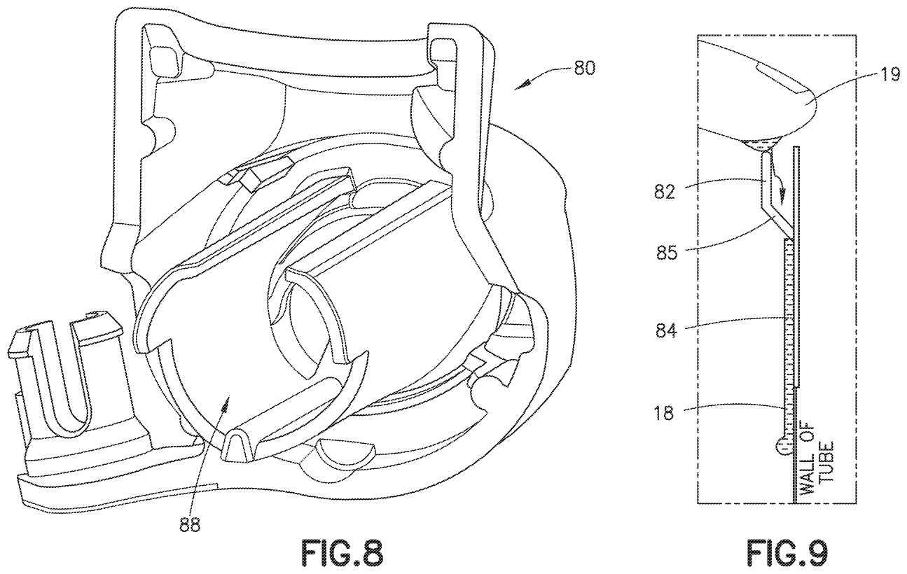

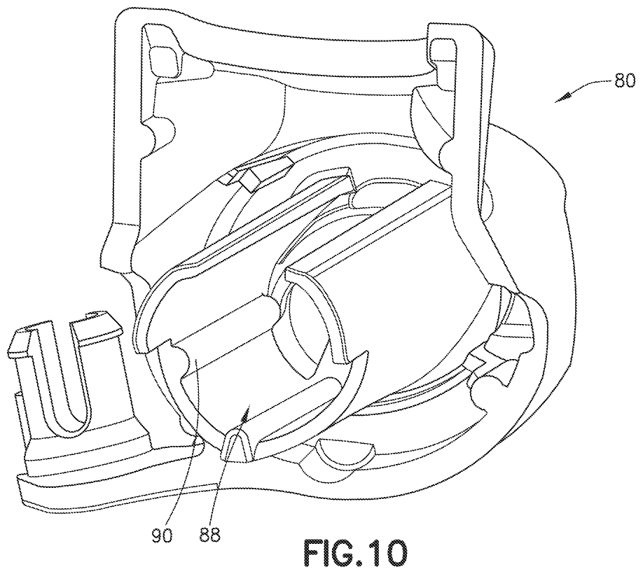

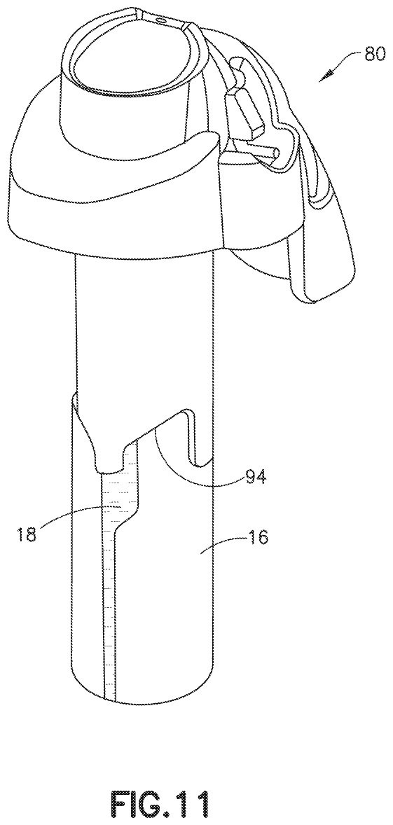

In another embodiment shown in FIGS. 8 and 9, an open channel flow path 88 is utilized with the blood collector attachment 80. In this configuration, a wider opening is provided for the blood collector attachment 80 to receive the blood sample 18. In another embodiment shown in FIG. 10, the open channel flow path 88 may also include at least one rib 90 that extends along the open channel flow path 88 to guide the flow of the blood sample 18 and minimize smearing of the blood sample 18 on the collection container 16. By optimizing the flow geometry from a capillary groove to an open guided flow minimizes the blood hang up in the blood collector attachment 80 and avoids pooling of blood in the blood collector attachment 80. In the open channel flow path 88, the blood flow may be slightly slower compared to the capillary channel, but there is no blood pooling and a reduced dead volume.

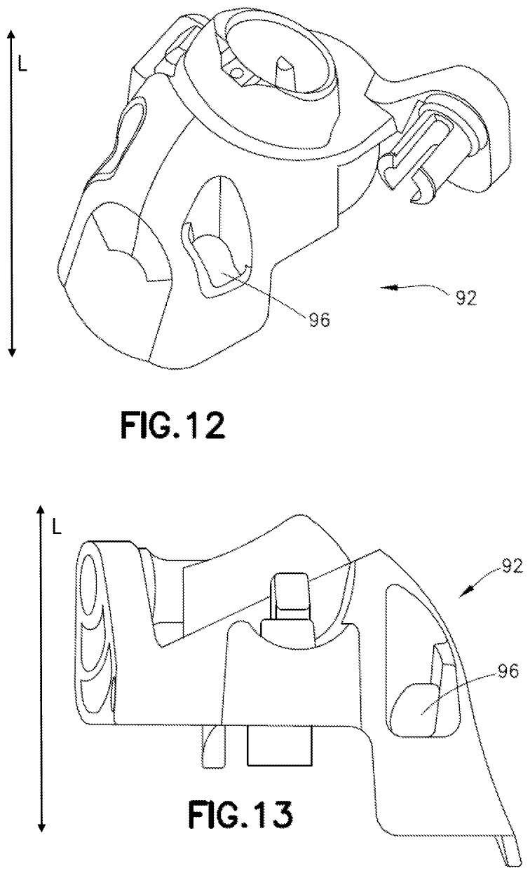

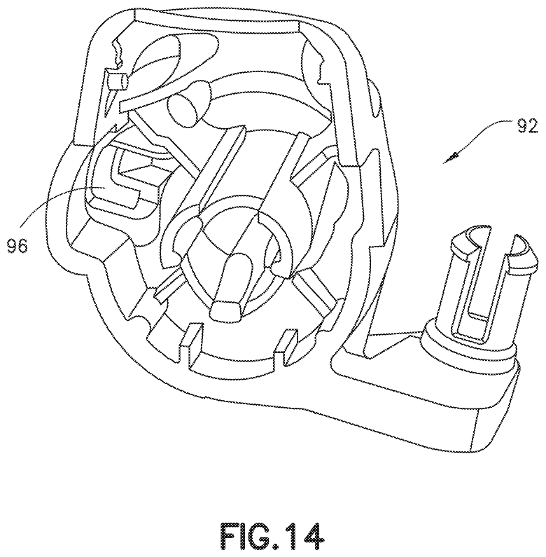

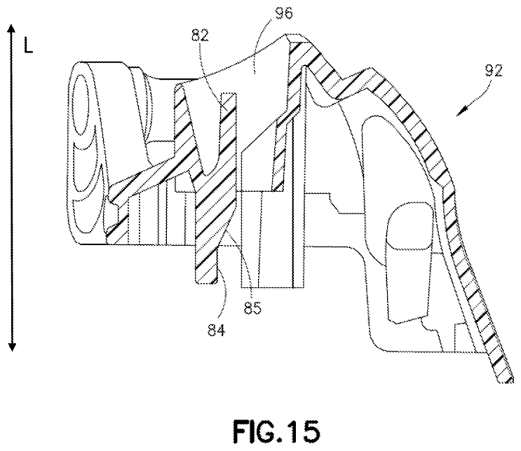

Cellular particles in the blood sample 18 like to stick to surfaces that they flow across, especially platelets that have an affinity to stick to foreign surfaces. The adhesion of these cellular particles may be minimized by minimizing the surface area-to-blood interaction. The guided flow path 84 in the blood collector attachment 80 may be optimized to minimize the surface area interaction with the blood sample 18. For example, in FIG. 6, the closed channel flow path 86 has a high surface area that creates a larger surface area-to-blood interaction. To reduce this interaction, a blood collector attachment 80 having an open channel flow path 88 as shown in FIG. 8 would assist in reducing the surface area that contacts the blood sample 18, thereby reducing the surface area-to-blood interaction. Further, the ribs 90 utilized with the open channel flow path 88 in FIG. 10 would further reduce the surface area-to-blood interaction. As shown in FIGS. 12-15, in another embodiment of the present disclosure, a “mini” blood collector attachment 92 that has reduced dimensions could be utilized to optimize the surface area-to-blood interaction by reducing the overall length L of the blood collector attachment 92, compared to the blood collector attachment 80, thereby reducing the surface area that the blood sample 18 travels across.

With reference to FIGS. 3 and 11, according to one embodiment of the present disclosure, the blood collector attachment 80 may also include features that assist in guiding and attaching the flow of the blood sample 18 from the blood collector attachment 80 into the collection container 16. In one example, at a bottom edge 94 of the blood collector attachment 80, the edge surfaces may be formed and configured to guide and enable attachment of the blood sample 18 to the collection container 16. This final attachment of the blood sample 18 into the collection container 16 establishes the complete flow path from the patients' finger 19 into the collection container 16. The bottom edge 94 of the blood collector attachment 80 may have a curvature that assists in guiding the blood sample 18 from the bottom edge 94 of the blood collector attachment 80 onto the inner surface of the collection container 16. In another embodiment of the present disclosure, the blood collector attachment 80 may also include vents 96 on a top surface of the blood collector attachment 80 to provide venting so the blood sample 18 flows freely down to the collection container 16 and does not get held up in the patient's finger 19 or the top of the blood collector attachment 80.

Current capillary blood collection workflow is complex, which requires a high skill level such as trained nurse or healthcare professionals. The complex workflow can introduce an increased amount of variability to the sample quality. Further, the open system collection also introduces an increased risk of blood exposure to healthcare workers. These deficiencies are addressed by blood collector attachment 80 of the present disclosure. Conventional blood sample collection involves multiple separate devices for lancing and collecting the blood sample. The collection tube needs to be precisely placed below the wound to catch the drop of blood coming out of the puncture site while squeezing the finger. This involves high skill and technique and potentially results in a high risk of blood exposure and contamination. The blood collector attachment 80 provides a closed system for blood collection minimizing any blood exposure risk.

With conventional collection, healthcare professionals tend to contact the wound with the tube in order to catch the drop of blood coming out of the wound. This type of collection is not the recommended method of collection but, due to the difficulty of capillary blood collection, sometimes healthcare professionals contact the wound with the collection tube. In the blood collector attachment 80 of the present disclosure, the position of the post member 82, which establishes the first drop attachment, has been optimized to ensure it does not contact the open wound, while at the same time catches the drop of blood coming out of the wound. This design reduces/eliminates any risk of infection.

With current conventional capillary collections, due to variability of squeezing and collection devices, the sample quality of blood sample collection is highly variable. The difficulty in aligning the blood collection tube to the wound results in smearing of blood on the finger and smearing on the top of the tube, which negatively impacts the sample quality. The blood collector attachment 80 minimizes these variables by ensuring a consistent flow path with minimal smearing of blood. In conventional collection, healthcare professionals also tend to scoop the blood from the wound using the tube, this can result in hemolysis. With the blood collector attachment 80, there is no blood scooping required to transfer the blood into the tube so there is no hemolysis risk.

While an embodiment of a capillary blood collection device is shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A device for obtaining a blood sample, the device comprising:
a holder for receiving a sample source, the holder having an actuation portion and a port;
a blood collector attachment removably connected to the holder; and
a collection container removably connectable to the blood collector attachment, the container defining a collection cavity,
wherein the blood collector attachment comprises a post member configured to direct the blood sample from a patient's finger to the collection container.

2. The device of claim 1, wherein the post member is positioned within the blood collector attachment so as to be directly beneath the patient's finger to make contact with the blood sample drawn from the patient's finger.

3. The device of claim 1, wherein a top edge of the post member is shorter than a top edge of the blood collector attachment.

4. The device of claim 1, wherein the post member is positioned within the blood collector attachment so as not to contact the patient's finger when held in the holder.

5. The device of claim 1, wherein the blood collector attachment further comprises a guided flow path connected to the post member for directing the blood sample from the post member to the collection container.

6. The device of claim 5, wherein the blood collector attachment further comprises a transition path provided between the post member and the guided flow path.

7. The device of claim 6, wherein the transition path has a curvature.

8. The device of claim 1, wherein a coating is applied to at least one of the blood collector attachment and the collection container to reduce adhesion of at least part of the blood sample thereto.

9. The device of claim 8, wherein the coating is applied to the post member.

10. The device of claim 8, wherein the coating is a protein coating.

11. The device of claim 10, wherein the protein coating comprises Bovine Serum albumin.

12. The device of claim 8, wherein the coating comprises EDTA.

13. The device of claim 1, wherein a bottom edge of the blood collector attachment has a curvature to direct the blood sample from the blood collector attachment to the collection container.

14. The device of claim 1, wherein at least one vent is defined in an upper surface of the blood collector attachment.

15. The device of claim 1, wherein the blood collector attachment defines an open channel flow path between the patient's finger and the collection container.

16. The device of claim 15, wherein the open channel flow path comprises at least one rib.

17.-33. (canceled)
